(19) United States
(12) Patent Application Publication
(10) Pub. No.: US 2005/0281749 A1
(43) Pub. Date: Dec. 22, 2005

(54) Sprayable compositions comprising a combination of pharmaceutical actives and an oily phase

(75) Inventors: Nathalie Willcox, Saint Vallier De Thiey (FR); Sandrine Orsoni, Mandelieu (FR); Laurent Fredon, Roquefort Les Pins (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA S.A.

(21) Appl. No.: 10/950,650

(22) Filed: Sep. 28, 2004

(30) Foreign Application Priority Data
Jun. 17, 2004 (FR) 04/06611

(51) Int. Cl. A61L 9/04; A61K 31/59

(52) U.S. Cl. 424/45; 514/167

(57) ABSTRACT

Sprayable, anhydrous and physically/chemically stable dermatological/pharmaceutical compositions, well suited for the treatment of a variety of dermatological disorders, notably psoriasis, contain: a) a therapeutically effective amount of a solubilized corticoid, notably dissolved clobetasol propionate; b) a therapeutically effective amount of a solubilized vitamin D derivative, notably dissolved calcitriol; and c) an oily phase which comprises one or more oils; formulated into d), a sprayable and topically applicable, dermatologically/pharmaceutically acceptable vehicle therefor.

### CROSS-REFERENCE TO PRIORITY APPLICATION

This application claims priority under 35 U.S.C. § 119 of FR 04/06611, filed Jun. 17, 2004, hereby expressly incorporated by reference and assigned to the assignee hereof.

### BACKGROUND OF THE INVENTION

1. Technical Field of the Invention

The present invention relates to anhydrous compositions in spray form comprising, as pharmaceutical active the combination of clobetasol propionate (corticoid) and calcitriol (vitamin D derivative) and an oily phase in a physiologically acceptable medium, to the process for preparing same and to dermatology applications thereof.

2. Description of Background and/or Related and/or Prior Art

The combination of active principles is not used conventionally in the treatment of dermatological ailments. The main difficulties encountered by one skilled in the art when combining two active principles are the problems of chemical instability and the interactions to which the active principles may initiate when they are present within the same formulation.

Few treatments therefore exist combining calcitriol and a corticoid. The reason for this is that vitamin D and its derivatives are instable in aqueous media and sensitive to acidic pH values, while the corticoids, and more particularly clobetasol propionate, are for their part sensitive to basic media. It was therefore not obvious for one skilled in the art to combine and to stabilize a vitamin D-type active and a corticosteroid within a single composition.

Calcitriol is a vitamin D analogue which is used to regulate the level of calcium in the body. Its use in the treatment of dermatological diseases was described in particular in U.S. Pat. No. 4,610,978 for the treatment of psoriasis. That patent suggests compositions combining calcitriol which may further comprise an amount of an anti-inflammatory such as a corticosteroid, but no specific embodiment combining calcitriol and corticosteroid is either described or tested for its efficacy.

FR-2,848,454, assigned to the assignee hereof, describes how combining calcitriol with a corticosteroid makes it possible to obtain a synergistic effect in the treatment of certain dermatological ailments such as psoriasis, atopic dermatitis, contact dermatitis and seborrheic dermatitis, though without proposing stable pharmaceutical compositions combining the two actives.

Furthermore, in the field of dermatology and of the formulation of pharmaceutical compositions, one skilled in the art seeks compositions which not only must be stable physically and chemically but also must make it possible to release the active and to promote its penetration through the skin layers in order to enhance its efficacy.

Pharmaceutical compositions must further exhibit good cosmetic qualities and must be preferably non-irritant.

Numerous topical compositions are currently in existence which comprise an active agent and allow its penetration into the skin to be promoted by virtue of the presence in particular of a high content of glycol penetration promoter. These compositions are formulated in the form of emulsions with a high fatty phase content which are commonly referred to as “lipocreams”, in the form of anhydrous compositions which are referred to as “ointments”, in the form of fluid compositions with a high content of volatile solvents, such as ethanol or isopropanol, which are intended for application to the scalp, and are also called “hair lotions”, or else in the form of viscous O/W emulsions, which are also called “O/W creams”.

The stabilization of a formulation comprising such a percentage of glycol makes it necessary to employ in the emulsion emulsifiers and stabilizers of glyceryl stearate or PEG 100 stearate type or else stabilizers or consistency factors of white wax or cetostearyl alcohol type which lead to the formation of a viscous cream, i.e., one whose viscosity is greater than 10 Pa.s (10,000 centipoises, measured with a Brookfield model LVDV II apparatus+spindle 4, at a rate of 30 revolutions/minute for 30 seconds and at a temperature of 25° C. +/−3° C.). This viscosity therefore makes the product difficult to apply. These compositions, however, exhibit, on the one hand, poor cosmetic acceptability owing to their viscosity and, on the other hand, risks of intolerance, brought about by the presence of high proportions of glycol. These high viscosities, moreover, make the formulations difficult to apply to the various parts of the body affected by the pathology. Consequently, the majority of existing treatments, in the form of creams or gels or ointments, necessitate the aid of a third person for their application to areas which are difficult to reach. The third person must therefore touch both the product containing the active and the psoriatic plaques, leading to a situation which is less than ideal from the standpoint of convenience of use and the third person's safety. One skilled in the art is also aware that non-compliance with the prescribed treatment for the reasons set out above is one of the principal causes of failure; the article “Patients with psoriasis and their compliance with medication” (Richard et al., J. Am. Acad. Dermatol., October 1999, p581-583) indicates that almost 40% of patients with a chronic disease such as psoriasis do not follow their treatment. It has been demonstrated that compliance by the patient with his or her treatment is directly linked to the characteristics of the vehicle of the applied composition. The article “Patients with psoriasis prefer solution and foam vehicles: a quantitative assessment of vehicle preference” (Housman et al., CUTIS, December 2002, Vol. 70, p327 to 332) indicates that psoriasis patients will give preference to a solution or a foam rather than to an unguent, cream or gel.

One skilled in the art therefore would improve these parameters by means of the invention hereinbelow described.

The closest prior art to the invention is WO 00/64450, which indicates a pharmaceutical composition containing a vitamin D analogue and a corticosteroid. All of the composition examples of that patent application combine solely calcipotriol and betamethasone dipropionate. The preferred compositions described in the application that allow the two actives to be stabilized are compositions in unguent form. These compositions therefore exhibit the drawbacks referred to above as far as convenience and ease of application are concerned. Study of this prior art in any event does not suggest sprayable compositions which hence are easy to apply, as described herein, with the actives, clobetasol propionate and calcitriol, solubilized and stable within the composition.

This is because, according to the prior art, either the existing treatments contain a high percentage of petroleum jelly, in order to promote occlusiveness and the penetration of the active, but have the disadvantage of being very greasy and sticky, or the compositions contain a high percentage of glycol penetration promoter in order to promote the penetration of the active, but are sticky and may give rise to problems of intolerance (“The critical role of the vehicle to therapeutic efficacy and patient compliance” Piacquadio et al., Journal of American Academy of Dermatology, August 1998).

### SUMMARY OF THE INVENTION

The problem which the present invention solves is therefore the design of a physically and chemically stable composition which allows the two actives calcitriol and clobetasol propionate, acting in synergy for the treatment of psoriasis, to be combined within a single composition, it being necessary also for the composition according to the invention to be easy to use and to have acceptable cosmetic properties for application to all of the areas of the body that may be affected by the pathology.

The term “physical stability” according to the invention refers to a composition which shows no change in its macroscopic appearance (phase separation, change in apparent color, etc.) or in its microscopic appearance (recrystallization of the active principle) after storage at temperatures of 4° C. and 40° C. for 2, 4, 8 and 12 weeks.

The term “chemical stability” according to the invention refers to a composition in which the active principle content remains stable after three months at ambient temperature and at 40° C. A stable active principle content signifies according to the invention that the content exhibits very little change relative to the initial content, in other words that the variation in active principle content at time T must not be less than 95% of the initial content at TO.

Thus, it has now surprisingly been found that compositions comprising, formulated into a pharmaceutically acceptable vehicle therefor:

a) a therapeutically effective amount of a corticoid in solubilized form, and more particularly clobetasol propionate;

b) a therapeutically effective amount of a vitamin D derivative in solubilized form, and more particularly calcitriol; and

c) an oily phase which comprises one or more oils; and being in anhydrous spray form, constitute compositions which ameliorate or avoid the above disadvantages and drawbacks of the prior art.

The compositions of the present invention are stable chemically and physically while allowing effective penetration of the active principles. They also exhibit very good acceptability and tolerance on the part of patients, owing to its spray formula, as described in the examples of the present invention. It therefore transpires that the compositions according to the invention are particularly suitable for the treatment of dermatological ailments, conditions and afflictions and more particularly are highly suited for the treatment of psoriasis.

The present invention accordingly features sprayable compositions comprising, in a pharmaceutically acceptable vehicle:

a) a therapeutically effective amount of clobetasol propionate in solubilized form;

b) a therapeutically effective amount of calcitriol in solubilized form;

c) an oily phase which comprises one or more oils.

### DETAILED DESCRIPTION OF BEST MODE AND SPECIFIC/PREFERRED EMBODIMENTS OF THE INVENTION

Advantageously the compositions according to the invention contain from 0.00001 to 0.1% by weight, relative to the total weight of the composition, of a vitamin D derivative active agent, preferably from 0.0001 to 0.001% by weight and more preferably from 0.0002 to 0.0005% by weight. The compositions according to the invention contain more particularly 0.0003% of calcitriol by weight, relative to the total weight of the composition.

Advantageously the compositions according to the invention contain from 0.0001 to 0.1% by weight, relative to the total weight of the composition, of a corticoid, preferably from 0.001 to 0.05% by weight. Preferred compositions according to the invention contain, more particularly, 0.025% or 0.05% of clobetasol propionate by weight, relative to the total weight of the composition.

An oily phase according to the invention is an oily phase suitable for a pharmaceutical composition.

The oily form is ideal for the psoriasis pathology. This liquid oily form makes it possible to provide the patient with the comfort of emollience without the drawbacks of applying a thick, very sticky and greasy ointment.

The selection and the ratio of the mixture of oils are determined as a function of their spreading powers and their chemical qualities. The selection of the oils which can be used in accordance with the invention is made such that the mixture thereof is clear and stable over time.

The oily phase of the compositions according to the invention may comprise, for example, vegetable, mineral, animal or synthetic oils, silicone oils and mixtures thereof.

As examples of mineral oils, mention may be made, for example, of liquid paraffins of various viscosities such as Primol 352, Marcol 82 and Marcol 152, sold by Esso.

As vegetable oils, mention may be made of sweet almond oil, palm oil, soya oil, sesame oil and sunflower oil.

As animal oils, mention may be made of lanolin oil, squalene, fish oil and mink oil.

As synthetic oils, mention may be made of an ester such as cetearyl isononanoate, for instance the product sold under the name Cetiol SN by Cognis France, diisopropyl adipate, for instance the product sold under the name Ceraphyl 230 by ISF, isopropyl palmitate, for instance the product sold under the name Crodamol IPP by Croda, isononyl isononanoate such as Dub Inin from Stearineries Dubois, and caprylic/capric triglyceride such as Miglyol 812, sold by Huls/Lambert Riviere.

As silicone oils, mention may be made of a dimethicone such as the product sold under the name Dow Corning 200 fluid, a cyclomethicone such as the product sold under the name Dow Corning 244 fluid by Dow Corning, or the product sold under the name Mirasil CM5 by SACI-CFPA. Mention may also be made of volatile silicone oils such as linear siloxanes and more preferably hexamethyldisiloxane. Mention may be made by way of example of the product sold by Dow Corning as DC Fluid 0.65cSt.

Preferably, the compounds constituting the oily phase of the compositions according to the invention are caprylic/capric triglycerides, sold under the name Miglyol 812, cetearyl isononanoate, sold under the name Cetiol SN, and cyclomethicone 5, sold under the name Mirasil CM5, which are used alone or in a mixture.

The rationale for selecting these preferred compounds is as follows:

Selection of caprylic/capric triglycerides:

Triglycerides are one of the components of the skin, forming part of the natural skin lipids together with the ceramides, cholesterol and the phospholipids. They are integrated into the deep layers of the epidermis and they compensate the skin's moisture loss. The protective barrier of the skin is regenerated specifically and durably. The “medium chain triglycerides”, of which Miglyol 812 is one, are composed of caprylic (C8) and capric (C10) fatty acids, which are derived from coconut oil or palm kernel oil.

The principal properties thereof are:

low-viscosity emollient, increasing spreading on the skin;

solvent for lipophilic active, rapidly penetrating the skin and promoting penetration of the active;

absence of greasy sensation on application, without leaving greasy residues.

Selection of cetaryl isononanoate:

Cetearyl isononanoate is an ester which has the particular feature of presenting a dry and soft feel to the skin.

Selection of cyclomethicone 5:

Cyclomethicone 5 is a volatile silicone oil which allows easy application to the skin and leaves a relatively dry feeling after application.

The judicious mixing and selection of the oils allow the patient to apply the product in sprayed form and allow, if desired, massage of the region to be treated, in contrast to a highly volatile sprayed product.

This also makes it possible to obtain a product which is totally oily but is much less greasy and sticky than unguents or ointments.

Advantageously, the compositions according to the invention contain from 50 to 99% by weight, relative to the total weight, of oily phase, preferably between 70 and 99% by weight and more preferably from 95 to 99% by weight.

The invention, accordingly, also features sprayable compositions comprising, in a pharmaceutically acceptable vehicle:

a) from 0.0001 to 0.1% of clobetasol propionate;

b) from 0.00001 to 0.1% of calcitriol;

c) from 50 to 99% of an oily phase which comprises one or more oils selected from among caprylic/capric triglycerides, cetearyl isononanoate and cyclomethicones.

More particularly the sprayable compositions preferred according to the present invention comprise, in a pharmaceutically acceptable vehicle:

a) from 0.001 to 0.05% of clobetasol propionate;

b) from 0.0002 to 0.0005% of calcitriol;

c) from 95 to 99% of an oily phase which comprises one or more oils selected from among caprylic/capric triglycerides, cetearyl isononanoate and cyclomethicones.

According to one preferred embodiment, the compositions according to the invention likewise comprise antioxidant compounds such as DL-α-tocopherol, butylated hydroxyanisole or butylated hydroxytoluene, superoxide dismutase, ubiquinol or certain metal chelating agents. The antioxidants preferably included in the compositions according to the invention are DL-α-tocopherol, butylated hydroxyanisole and butylated hydroxytoluene.

The compositions according to the invention may likewise comprise surfactants. The surfactants which can be included according to the invention are of anionic surfactant type, such as carboxylates, and particularly soaps, alkylarylsulfonates, alkyl ether sulfates, alkyl sulfates and alcohol sulfates. More particularly, the anions of these surfactants are coupled to a cation such as the metallic cations of sodium or of potassium. The preferred surfactants according to the invention are also surfactants of polysorbate and poloxamer type.

Preferably, the surfactants according to the present invention are sodium lauryl sulfate, polysorbate 80 (Tween 80 from Uniqema) and poloxamer 124 (Synperonic PEL44 from Uniqema).

The compositions according to the invention may likewise comprise penetration promoters. The penetration promoters which can be included according to the invention are of alcohol type, such as ethanol, or of glycol type, such as 1,2-propanediol, known under the name propylene glycol and sold by Dow Chemical.

The pharmaceutical compositions according to the invention may further comprise inert additives or combinations of these additives, such as:
wetting agents;
flavor enhancers;
preservatives;
stabilizers;
moisture regulators;
pH regulators;
osmotic pressure modifiers;
emulsifiers;
UV-A and UV-B filters;
penetration promoters; and
synthetic polymers.

As will be appreciated, one skilled in the art will take care that any compound or compounds for addition to the subject compositions will be selected such as not to alter, or substantially alter, the advantageous properties intrinsically associated with the present invention, as a result of the intended addition.

The compositions according to the invention are more particularly suited for a regime or regimen for the treatment of the skin and the mucosae and are sprayable and suitable for packaging in spray form.

The spray exhibits numerous advantages over conventional forms, such as the ease with which the formula can be delivered into the areas of the body that are very difficult to treat, the possibility of ready control of the dose delivered, or the absence of contamination during use.

The compositions according to the invention are therefore administered in the form of a sprayable composition. These compositions may be prepared by conventional formulating means which are known to one skilled in the art. For example, the compositions may be sprayed by a mechanical sprayer which pumps the composition within a container, flask or equivalent. Similarly, the compositions may be propelled by means of a gas, as is well known to this art. Conventional propellants such as air or hydrocarbons are effective provided that they do not interfere with the composition. The composition passes through a nozzle which may be pointed directly at the place where application is desired. The nozzle may be selected so as to apply the composition in the form of a vaporization or of a jet of droplets, according to the techniques known to the art. Depending on the pharmaceutical active selected, the spraying mechanism must be capable of always delivering the same amount of active. The mechanisms which allow the amount of composition to be delivered by the spray to be controlled are likewise known to this art. For example, the amount of propellant may be calculated so as to propel the exact amount of product desired. For the compositions according to the invention it is possible to use a metering vaporizer flask whose application surface characteristics and dose characteristics are controlled and reproducible. For example, the vaporizer may be composed of a flask fitted with a metering valve.

The compositions of the present invention are chemically and physically stable while allowing effective penetration of the active principles. They also exhibit very good acceptability and tolerance on the part of patients, owing to its spray formula, as described in the examples of the present invention. It is therefore found that the compositions according to the invention are particularly suitable for the treatment of dermatological ailments.

The present invention hence also features the formulation of a composition according to the invention for producing a medicinal product suited for the treatment of:

dermatological ailments linked to a keratinization disorder relating to differentiation and to proliferation, especially common acne, comedo-type acne, polymorphic acne, acne rosacea, nodulocystic acne, acne conglobata, senile acnes and secondary acnes such as solar acne, drug-induced acne or occupational acne,

ichthyoses, ichthyosiform conditions, Darrier's disease, palmoplantar keratodermas, leukoplakia and leukoplakiform conditions, cutaneous lichen or mucosal (oral) lichen,

dermatological ailments or disorders having an inflammatory immunoallergic component, with or without a cell proliferation disorder, especially cutaneous psoriasis, mucosal psoriasis or ungual psoriasis, psoriatic rheumatism, cutaneous atopy, such as eczema, respiratory atopy or gingival hypertrophy,

dermal or epidermal proliferations, benign or malignant, of viral or other origin, especially common warts, flat warts, verruciform epidermodysplasia, oral or florid papillomatoses and T lymphoma,

proliferations which may be induced by ultraviolet radiation, especially basal cell epithelioma and spinocellular epithelioma,

precancerous skin lesions, especially keratoacanthomas,

immune dermatoses, especially lupus erythematosus,

bullous immune diseases,

collagen diseases, especially scleroderma,

dermatological or systemic ailments or disorders having an immunological component,

skin disorders due to exposure to UV radiation, skin aging, light-induced or chronological, or actinic keratoses and pigmentations, or any pathologies associated with chronological ageing or actinic aging, especially xerosis,

sebaceous function disorders, especially hyperseborrhoea of acne, simple seborrhoea or seborrhoic dermatitis,

cicatrization disorders or stretchmarks,

pigmentation disorders, such as hyperpigmentation, melasma, hypopigmentation or vitiligo,

lipid metabolism ailments or disorders, such as obesity, hyperlipidemia, non-insulin-dependent diabetes or syndrome X,

inflammatory ailments such as arthritis,

cancerous or precancerous conditions,

alopecia of various origins, especially alopecia due to chemotherapy or to radiation,

immune system disorders, such as asthma, type 1 sugar diabetes, multiple sclerosis, or other selective dysfunctions of the immune system, or

ailments or disorders of the cardiovascular system such as arteriosclerosis or hypertension.

In one preferred embodiment of the composition, it will contain 0.025% or 0.05% of clobetasol 17-propionate and 0.0003% of calcitriol and will be used for formulating a medicinal product suited for treating psoriasis.

The examples which follow show without limitation formulation examples of the composition according to the invention and also results for chemical and physical stability.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Stability of Calcitriol in Various Excipients

The example below describes the stability data for calcitriol in the various excipients preferred for the composition according to the invention, including caprylic/capric triglycerides and cetearyl isononanoate.

a) Stability of calcitriol in Miglyol 812 (caprylic/capric triglycerides):

Solution of calcitriol 30 ppm in qs 100% of Miglyol 812 in the presence of 0.4% of BHT. HPLC assay technique against reference substance.

At the starting time (TO) the composition is considered to contain 100% of calcitriol.

Concentration of calcitriol measured in % relative to TO:

| Stability conditions | T 2 weeks | T 4 weeks |
|---|---|---|
| +4° C. | 98.3% | 105.2% |
| AT | 95.1% | 98.0% |
| +40° C. | 91% | 93.8% |

b) Stability of calcitriol in Cetiol SN (cetearyl isononanoate):

Solution of calcitriol 30 ppm in qs 100% of Cetiol SN (cetearyl isononanoate) in the presence of 0.4% of BHT.

HPLC assay technique against reference substance.

At the starting time (TO) the composition is considered to contain 100% of calcitriol.

Concentration of calcitriol measured in % relative to TO:

| Stability conditions | T 2 weeks | T 4 weeks |
|---|---|---|
| +4° C. | 98.6% | 98.1% |
| AT | 98.7% | 98.4% |
| +40° C. | 99.0% | 98.9% |

### EXAMPLE 2

### Process for Preparing Compositions According to the Invention

Compositions according to the present invention are prepared at ambient temperature, under a fume hood and in non-actinic light.

Introduce the antioxidant, the Calcitriol and the oil into a flask.

Carry out stirring until the calcitriol is completely dissolved.

Then add the Clobetasol Propionate.

Continue stirring until the clobetasol propionate is dissolved.

When the two actives are completely dissolved, introduce the remaining ingredients of the formula in succession.

Maintain under stirring until the mixture is perfectly homogeneous.

### EXAMPLE 3

| INGREDIENTS | % |
|---|---|
| CYCLOMETHICONE 5 | qs 100 |
| MEDIUM CHAIN TRIGLYCERIDES | 40 |
| CALCITRIOL | 0.0003 |
| CLOBETASOL 17-PROPIONATE | 0.025 |
| DL-ALPHA-TOCOPHEROL ACETATE | 1 |

The procedure is that described in Example 2.

A colorless liquid solution is obtained.

### EXAMPLE 4

| INGREDIENTS | % |
|---|---|
| CYCLOMETHICONE 5 | qs 100 |
| MEDIUM CHAIN TRIGLYCERIDES | 40 |
| CALCITRIOL | 0.0003 |
| CLOBETASOL 17-PROPIONATE | 0.025 |
| 1,2-PROPANEDIOL | 10 |
| ALMOND OIL | 5 |

The procedure is that described in Example 2.

A very slightly yellow liquid solution is obtained.

### EXAMPLE 5

| INGREDIENTS | % |
|---|---|
| CETEARYL ISONONANOATE | qs 100 |
| MEDIUM CHAIN TRIGLYCERIDES | 40 |
| CALCITRIOL | 0.0003 |
| CLOBETASOL 17-PROPIONATE | 0.025 |
| DIMETHICONE 200, 20 CST | 10 |
| ALMOND OIL | 5 |

The procedure is that described in Example 2.

A very slightly yellow liquid solution is obtained.

### EXAMPLE 6

| INGREDIENTS | % |
|---|---|
| MEDIUM CHAIN TRIGLYCERIDES | qs 100 |
| CYCLOMETHICONE 5 | 45 |
| CALCITRIOL | 0.0003 |
| CLOBETASOL 17-PROPIONATE | 0.025 |
| DIMETHICONE 200, 20 CST | 10 |
| ALMOND OIL | 5 |

The procedure is that described in Example 2.

A very slightly yellow liquid solution is obtained.

### EXAMPLE 7

### Physical Stability of the Composition According to Example 6

The physical stability of the formulations is measured by macroscopic and microscopic observation of the formulation at ambient temperature, at 4° C. and at 40° C. after 2, 4, 8 and 12 weeks.

At ambient temperature macroscopic observation allows the physical integrity of the products to be guaranteed and microscopic observation makes it possible to verify that there is no recrystallization of the solubilized active.

At 4° C. microscopic observation verifies the non-recrystallization of the solubilized actives.

At 40° C. macroscopic observation verifies the integrity of the end product.

Specifications at TO:

Macroscopic Appearance

Colourless or very slightly yellow liquid spray.

Microscopic Appearance

Absence of crystals of calcitriol and of clobetasol 17-propionate.

| | Time |
|---|---|
| Stability conditions↓ | T 15 days |
| AT | Conforms to specifications |
| +4° C. | Conforms to specifications |
| 40° C. | Conforms to specifications |

### EXAMPLE 8

### Chemical Stability of the Actives within the Composition According to Example 6

Stability of the Calcitriol:

The active is assayed by internal calibration in HPLC.

The results are expressed in % recovery relative to the theoretical value.

| | Time |
|---|---|
| Stability conditions↓ | T 15 days |
| AT | 96.6% |
| 40° C. | 105.6% |

Stability of the clobetasol 17-propionate:

Assay of the active by internal calibration in HPLC.

The results are expressed in % recovery relative to the theoretical value.

| | Time |
|---|---|
| Stability conditions↓ | T 15 days |
| AT | 98.2% |
| +40° C. | 98.5% |

### EXAMPLE 9

| INGREDIENTS | % |
|---|---|
| MEDIUM CHAIN TRIGLYCERIDES | qs 100 |
| CETEARYL ISONONANOATE | 45 |
| CALCITRIOL | 0.0003 |
| CLOBETASOL 17-PROPIONATE | 0.025 |
| ALMOND OIL | 5 |

The procedure is that described in Example 2.

A very slightly yellow liquid solution is obtained.

### EXAMPLE 10

### Physical Stability of the Compositions According to Example 9

The physical stability of the formulations is measured by macroscopic and microscopic observation of the formulation at ambient temperature, at 4° C. and at 40° C. after 2, 4, 8 and 12 weeks.

At ambient temperature macroscopic observation allows the physical integrity of the products to be guaranteed and microscopic observation makes it possible to verify that there is no recrystallization of the solubilized active.

At 4° C. microscopic observation verifies the non-recrystallization of the solubilized actives.

At 40° C. macroscopic observation verifies the integrity of the end product.

Specifications at TO:

Macroscopic Appearance

Colourless or very slightly yellow liquid spray.

Microscopic Appearance

Absence of crystals of calcitriol and of clobetasol 17-propionate.

| | Time |
|---|---|
| Stability conditions↓ | T 15 days |
| AT | Conforms to specifications |
| +4° C. | Conforms to specifications |
| 40° C. | Conforms to specifications |

### EXAMPLE 11

### Chemical stability of the actives within the Composition According to Example 9

Stability of the Calcitriol:

The active is assayed by internal calibration in HPLC.

The results are expressed in % recovery relative to the theoretical value.

| | Time |
|---|---|
| Stability conditions↓ | T 15 days |
| AT | 101.4% |
| +40° C. | 102.9% |

Stability of the clobetasol 17-propionate:

Assay of the active by internal calibration in HPLC.

The results are expressed in % recovery relative to the theoretical value.

| | Time |
|---|---|
| Stability conditions↓ | T 15 days |
| AT | 97.8% |
| +40° C. | 99% |

### EXAMPLE 12

| INGREDIENTS | % |
|---|---|
| CYCLOMETHICONE 5 | qs 100 |
| MEDIUM CHAIN TRIGLYCERIDES | 45 |
| CALCITRIOL | 0.0003 |
| CLOBETASOL 17-PROPIONATE | 0.025 |
| DIMETHICONE 200, 20 CST | 10 |

The procedure is that described in Example 2.

A colourless liquid solution is obtained.

Specifications at TO:

Macroscopic Appearance

Colourless or very slightly yellow liquid spray.

Microscopic Appearance

Absence of crystals of calcitriol and of clobetasol 17-propionate.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A sprayable, anhydrous and physically/chemically stable dermatological/pharmaceutical composition, comprising:
a) a therapeutically effective amount of a solubilized corticoid;
b) a therapeutically effective amount of a solubilized vitamin D derivative; and
c) an oily phase which comprises one or more oils; formulated into d), a sprayable and topically applicable, dermatologically/pharmaceutically acceptable vehicle therefor.

2. The sprayable, anhydrous dermatological/pharmaceutical composition as defined by claim 1, said corticoid comprising clobetasol propionate.

3. The sprayable, anhydrous dermatological/pharmaceutical composition as defined by claim 2, said vitamin D derivative comprising calcitriol.

4. The sprayable, anhydrous dermatological/pharmaceutical composition as defined by claim 3, said oily phase comprising one or more oils selected from the group consisting of caprylic/capric triglycerides, cetearyl isononanoate and cyclomethicones.

5. The sprayable, anhydrous dermatological/pharmaceutical composition as defined by claim 4, comprising:
a) from 0:0001 to 0.1% of clobetasol propionate;
b) from 0.00001 to 0.1% of calcitriol;
c) from 50 to 99% of an oily phase which comprises one or more oils selected from the group consisting of caprylic/capric triglycerides, cetearyl isononanoate and cyclomethicones.

6. The sprayable, anhydrous dermatological/pharmaceutical composition as defined by claim 5, comprising:
a) from 0.001 to 0.05% of clobetasol propionate;
b) from 0.0002 to 0.0005% of calcitriol;
c) from 95 to 99% of an oily phase which comprises one or more oils selected from the group consisting of caprylic/capric triglycerides, cetearyl isononanoate and cyclomethicones.

7. The sprayable, anhydrous dermatological/pharmaceutical composition as defined by claim 1, further comprising an antioxidant.

8. The sprayable, anhydrous dermatological/pharmaceutical composition as defined by claim 7, said antioxidant being selected from the group consisting of DL-α-tocopherol, butylated hydroxyanisole and butylated hydroxytoluene.

9. The sprayable, anhydrous dermatological/pharmaceutical composition as defined by claim 1, further comprising a surfactant.

10. The sprayable, anhydrous dermatological/pharmaceutical composition as defined by claim 9, said surfactant being selected from the group consisting of sodium lauryl sulfate, poloxamers and polysorbates.

11. The sprayable, anhydrous dermatological/pharmaceutical composition as defined by claim 1, further comprising a penetration promoter.

12. The sprayable, anhydrous dermatological/pharmaceutical composition as defined by claim 11, said penetration promoter comprising 1,2-propanediol or ethanol.

13. A regime or regimen for preventing or treating dermatological conditions associated with a keratinization disorder relating to differentiation and to proliferation, common acne, comedo-type acne, polymorphic acne, acne rosacea, nodulocystic acne, acne conglobata, senile acne, secondary acne, solar acne, drug-induced acne or occupational acne; ichthyoses, ichthyosiform conditions, Darrier's disease, palmoplantar keratodermas, leukoplakia and leukoplakiform conditions, cutaneous lichen or mucosal (oral) lichen; dermatological conditions having an inflammatory immunoallergic component, with or without a cell proliferation disorder, cutaneous psoriasis, mucosal psoriasis or ungual psoriasis, psoriatic rheumatism, cutaneous atopy, eczema, respiratory atopy or gingival hypertrophy; dermal or epidermal proliferations, benign or malignant, of viral or other origin, common warts, flat warts, verruciform epidermodysplasia, oral or florid papillomatoses and T lymphoma; proliferations induced by ultraviolet radiation, basal cell epithelioma and spinocellular epithelioma; precancerous skin lesions, keratoacanthomas; immune dermatoses, lupus erythematosus; bullous immune diseases; collagen diseases, scleroderma; dermatological or systemic disorders having an immunological component; skin disorders due to exposure to UV radiation, skin aging, light-induced or chronological, or actinic keratoses and pigmentations, or any pathologies associated with chronological aging or actinic aging, xerosis; sebaceous function disorders, hyperseborrhoea of acne, simple seborrhoea or seborrhoic dermatitis; cicatrization disorders or stretchmarks; pigmentation disorders, hyperpigmentation, melasma, hypopigmentation or vitiligo; lipid metabolism ailments disorders, obesity, hyperlipidemia, non-insulin-dependent diabetes or syndrome X; inflammatory disorders, arthritis; cancerous or precancerous conditions; alopecia of various origins, alopecia due to chemotherapy or to radiation; immune system disorders, asthma, type 1 sugar diabetes, multiple sclerosis, or other selective dysfunctions of the immune system; or disorders of the cardiovascular system, arteriosclerosis or hypertension, comprising spraying onto the affected skin area of an individual in need of such treatment, a thus effective amount of the sprayable, anhydrous dermatological/pharmaceutical composition as defined by claim 1.

14. A regime or regimen for the treatment of psoriasis, comprising spraying onto the affected area of the skin of an individual afflicted with psoriasis, of the sprayable, anhydrous dermatological/pharmaceutical composition as defined by claim 1.

15. A spray dispenser comprising a housing confining a sprayable, anhydrous dermatological/pharmaceutical composition as defined by claim 1, and a pumping element for mechanically spraying said composition out of said housing.

16. A spray dispenser comprising a housing confining a sprayable, anhydrous dermatological/pharmaceutical composition as defined by claim 1, and a gaseous propellant for spraying said composition out of said housing.

17. The spray dispenser as defined by claim 15, further comprising a metering element for spraying/delivering essentially the same amount of said composition.

18. The spray dispenser as defined by claim 16, comprising an amount of propellant effective for spraying/delivering essentially the same amount of said composition.
